(19) United States
(12) Patent Application Publication
(10) Pub. No.: US 2004/0015074 A1
(43) Pub. Date: Jan. 22, 2004

(54) Radio frequency coil for resonance imaging analysis of pediatric patients

(76) Inventor: Ravi Srinivasan, Richmond Heights, OH (US)

Correspondence Address: Mark D. Saralino, Renner, Otto, Boisselle & Sklar, LLP, Nineteenth Floor, 1621 Euclid Avenue, Cleveland, OH, 44115, US

(21) Appl. No.: 10/440,054

(22) Filed: May 16, 2003

Related U.S. Application Data
(60) Provisional application No. 60/381,160, filed on May 16, 2002.

(51) Int. Cl. A61B 5/055; G01V 3/00

(52) U.S. Cl. 600/422; 324/318

(57) ABSTRACT

A radio frequency head coil for resonance imaging analysis of pediatric patients is disclosed. The head coil includes an end cap, an end ring, a plurality of legs connecting the end cap to the end ring, wherein the plurality of legs are spaced apart azimuthally by 45 degrees, and a plurality of drive points, wherein the drive points are bridged using a push-pull configuration.

### CROSS REFERENCE TO RELATED APPLICATIONS

This Application claims priority from U.S. Provisional Application Serial No. 60/381,160 filed May 16, 2002, which is incorporated herein by reference.

### TECHNICAL FIELD

The present invention relates to a magnetic resonance imaging (MRI) system and a life sustaining incubator system used for ill neonates. Specifically, the invention relates to a radio-frequency (RF) coil and method for use in such systems. More specifically, the invention relates to an RF coil and method for use with the MRI compatible incubator (MRCI) in the MRI system.

### BACKGROUND

NMR or MRI

In MRI systems or nuclear magnetic resonance (NMR) systems, a static magnetic field is applied to the body under investigation to define an equilibrium axis of magnetic alignment in the region of the body under investigation. An RF field is applied in the region being examined in a direction orthogonal to the static field direction, to excite magnetic resonance in the region, and resulting RF signals are detected and processed. Generally, the resulting RF signals are detected by RF coil arrangements placed close to the body. See for example, U.S. Pat. No. 4,411,270 to Damadian and U.S. Pat. No. 4,793,356 to Misic et al. Typically, these coils are either surface type or volume type, depending on the application and are used to transmit RF and receive NMR signals from the region of interest (ROI).

A further increase in signal to noise ratio (S/N) can be realized with the use of quadrature coils over the conventional linear coil design. See for example U.S. Pat. No. 4,467,282 to Siebold and U.S. Pat. No. 4,707,664 to Fehn. Also see U.S. U.S. Pat. No. 4,783,641 to Hayes [1] and U.S. Pat. No. 4,751,464 to Bridges [2], for highly homogeneous, quadrature volume coils commonly referred to as the birdcage and transverse electromagnetic (TEM) wave resonators respectively, in the NMR community.

Neonate Incubator

Incubators are commonly used in hospitals in the neonatal intensive care units (NICUs) as life sustaining devices for the ill neonate. These incubators help to maintain the micro-environment of the ill neonate with high levels of temperature (up to 39 deg C.), humidity (up to 85%) and oxygen (up to 75%) levels prescribed by the doctor and demanded by the patient for patient survival. Generally, mildly ill neonates are transported to the MRI scanner and placed inside the super cooled MR system for diagnosis. No effort is made to maintain the micro-environment surrounding the patient to the original NICU conditions inside the incubator. This was due to the unavailability of an incubator system that was MRI compatible.

Recently, a submission was made to the European patent office patent office (see EP 01 109 195.6, filed Apr. 12, 2001) and a worldwide patent application (filed Apr. 12, 2001) by Lonekker—Lammers et al [3, 4] for an incubator/transporter system that is MRI compatible. With this MRCI, safe transport is possible between the NICU and the MRI sections. Further, with this incubator the neonate is left untouched inside the incubator when scanned inside the MRI system. Once the MRI scan is complete, the patient is transported back to the NICU in the same incubator system. Thus this setup obviates for the need to disturb the patient leaving all of the incubator settings (temperature, humidity, oxygen) in tact.

In summary, this MRCI performs-similar to the conventional NICU transport incubator with the added feature of MRI compatibility. The added feature provides the clinicians the necessary diagnostic information which may lead to prompt clinical/pharmacological/surgical interventions, which in turn save precious lives.

MRI as we know is a diverse imaging tool commonly used in the diagnosis/prognosis of illnesses in the pediatric population [5]. MRI diagnosis however depends on the image quality. For a particular field strength, high image qualities over the ROI can be achieved with a high S/N RF coil.

Birdcage Coil

The birdcage coil is well known in the art is described in reference [1]. The coil includes two end rings connected by several straight segments referred to as legs. This coil has several resonance modes, of interest is the principal k=1 mode for homogeneous imaging. The principal mode has two linear modes, oriented orthogonal to one another. The outputs from these modes can be combined using analog circuitry or digitally combined in the receiver system. The birdcage provided 41% improvement in S/N and expended one-half the power over the conventional linear coil.

In addition, owing to the sinusoidal currents in the coil periphery the birdcage provided a highly homogeneous B field in the transverse planes (XY) inside the coil, ideal for imaging (whole-body, head, knee, wrist, etc. for adults). The B field profile along the coil axis, however, mimicked a Gaussian distribution with maximum at the coil center.

This distribution was improved over the adult head with an end-capped design of Hayes [6], which provided a more uniform distribution toward the top of the head. This field distribution for this coil design along the coil axis toward the open end fell off like a conventional birdcage, which was ideal for imaging the adult head.

At present, the neonates are imaged using adult coils inside the super cooled MRI scanner without the incubator. Should the incubator be used inside the MRI scanner, the S/N of the MRI experiment will greatly suffer when larger adult size coils are used to encompass the incubator. Specialty, RF coils must be used to attain optimum S/N and imaging resolution. Further, the coil must withstand the harsh environment (high temperature, high levels of humidity and oxygen) within the incubator. In addition, the design must allow rapid positioning/removal over the patient pre/post MR scan and must allow placement of endo-tracheal tubes (ett) and similar devices (ventilator tubes) attached to the patient.

### SUMMARY OF THE INVENTION

The general purpose of this invention is to provide an RF coil with improved signal-to-noise ratio (S/N) capable of high resolution imaging over the head of the pre-, term and post-term newborns. A further purpose is to provide a RF coil that is suitable for use with an MR compatible incubator.

Our disclosure is intended to enhance the S/N of the RF coil system over the pre-, term- and post-term pediatric head (age 0-3 months). In addition, this disclosure is intended for use within the MR compatible incubator (MRCI) without significantly sacrificing its performance or that of the incubator.

By using the coil within the incubator, high image S/N and thereby high image quality can be realized in reasonable scan times. With the improved S/N, one can increase the imaging resolution or reduce the scan time thereby reducing patient risks while concomitantly increasing throughput in an MR scanner.

According to one aspect of the invention, the invention is directed to a head coil that includes an end cap, an end ring, a plurality of legs connecting the end cap to the end ring, wherein the plurality of legs are spaced apart azimuthally by 45 degrees, and a plurality of drive points, wherein the drive points are bridged using a push-pull configuration.

### BRIEF DESCRIPTION OF DRAWINGS

These and further features of the present invention will be apparent with reference to the following description and drawings, wherein:

FIG. 1 is a block diagram of a system incorporating a coil in accordance with the present invention;

FIG. 2 is an isometric view of an MRI system incorporating a coil in accordance with an embodiment of the present invention.

FIG. 3 is an isometric view of a birdcage coil in accordance with the present invention;

FIG. 4 is a cross sectional view of the birdcage coil of FIG. 3.

FIG. 5 is a planar schematic view of the coil of FIG. 3 showing the capacitor values of the coil;

FIG. 6 is a schematic view of the coil of FIG. 3 illustrating the four-point feed connections;

FIG. 7 is an isometric view of an MRI system incorporating a coil in accordance with another embodiment of the present invention.

### DISCLOSURE OF INVENTION

A primary object of the invention is to provide an RF coil with high S/N over the pediatric head. A secondary object is to provide an RF coil with high S/N that is safe for use with an incubator inside the MRI.

The first embodiment is directed to a head RF coil specific for use with pre-, term and post-term born newborns and for use on infants, for example, up to 3 months of age. Further embodiments can be realized for imaging the torso (heart, liver, spleen, etc.) and the upper/lower extremity of the infant.

Patient Transport

Generally, the patient will be transported in the MRCI from the NICU to the MRI room. Then the RF coil will be slid inside the MR compatible incubator and over the patient's head prior to the MRI scan. Once the scan has finished the RF coil either will be slid back or removed from the incubator, prior to transporting the patient back to the NICU. This way the patient is left untouched and the micro-environment of the incubator with prescribed temperature, humidity and oxygen levels is left virtually un-disturbed.

Power Deposition

Note, the pediatric patients planned to be scanned with the coil in the present invention are very tiny and weigh anywhere between 1200 to 5000 grams. The FDA has a limit for RF power deposition of not to exceed 3.2 watts per kilogram over the adult head. For a 1200 gram baby, the head will weigh approximately 480 grams (roughly 40% of body weight). Should we scale the FDA guideline for adults to the pediatric population, one must not use more than 1.28 watts over the head for a patient weighing 1200 grams. A smaller coil will be more efficient and will expend less RF power than the adult head coil. This necessitates the development of a scaled down custom head coil for the tiny pediatric patient.

Note, a custom RF coil for the newborn brain will result in an efficient operation, will use a fraction of the power currently used by adult head coils, will minimize image artifacts from neighboring anatomies (neck, chest, shoulders) and will provide a high S/N and resolution sought by the clinician for effective diagnosis of the illnesses. Further, the additional S/N can be used to reduce the scan time and therefore patient stay inside the MRI scanner (away from the NICU). Note with the coil mentioned in this application, we are able to achieve optimum imaging over the pediatric brain below the FDA guidelines mentioned above.

RF Coil Design Considerations

1) All coils must withstand the relatively high temperature (up to 39 deg C. as opposed to room temperature of 25 deg C.), high levels of humidity (up to 85%) and varying amounts of enriched oxygen (up to 75%) inside the incubator.

2) RF coils must be large enough to accommodate the sick patient connected to one or more tubes for incubation.

3) Coils must provide high S/N over the imaging field of view (FOV).

4) Coils must not impair the incubator functions and must be easy to use.

To address the first point, plastic composites chosen to build the RF coil were thermally stable, had low moisture absorption coefficient and did not react with oxygen. Coil enclosures were machined with close tolerances to attain a press-fit between male and female components. There were no holes in the RF coil that exposed the coil to the patient, this was necessary to eliminate leakage of oxygen and moisture (caused due to high humidity) from reaching the high voltage parts (tuning and matching capacitor) of the coils. The front end ring and end cap of the coil were sealed with an encapsulant safe for use with electrical components and suitable for use with MRI.

The encapsulant chosen had high dielectric strength (>400 volts per mil, 1 mil=0.001″), high dielectric constant, low thermal expansion coefficient low water absorption, high volume resistivity and must cure at room temperature within a few days. In addition, the encapsulant must not significantly affect coil tuning or coil Q values. For example, we evaluated three encapsulants which satisfied all of the above criteria from Lord Chemicals (urethane based UR-324, silicon based SC-102, epoxy based ES-100). The shift in coil tuning was less than 1 MHz at the NMR frequency of 64 MHz and the coil Q remained virtually the same. Further, we compared performances of two near identical coils with one sealed. Virtually no noticeable difference in coil performance was seen. S/N of both coils were within 2% which was within the MRI equipment tolerances for S/N. In all cases, MR images were noticed for artifacts caused due to the encapsulant primarily on weak signal scans that used short T2 (TE 3-4 ms). Very little or no artifacts were witnessed with these encapsulants.

The legs of the coil were covered with fiberglass tubes capable of withstanding the environment of the incubator. All of the electronics in the vicinity of the end-cap were sealed including the cable outlets pointing toward the rear of the incubator (see FIG. 3).

To address the second point, the dimension of the coil was chosen to include 95th percentile of the patient population up to 3 months of age, for example. Extra space was provided in the anterior section toward the front end to allow room for endo-tracheal and ventilator tubes to be connected to the patient during the scan (see FIG. 3).

To address the third point, the birdcage design was chosen to provide a high S/N and high degree of RF homogeneity over the pediatric head owing to its quadrature operation (√2 improvement in S/N) and sinusoidal current distribution.

To address the fourth point, the coil was designed with virtually no perturbation of air flow to the patients face alongside the length of the patient. The head coil of FIG. 3, will be slid in to the incubator via a rear flap (not shown) prior to the scan and removed after the MRI scan without disturbing the patient. The coil must be held still during scanning to eliminate ghosting artifacts caused due to vibration. Our coil will be held in place during the scan with rubber bushings attached to the coil using the incubator flap. The easy ON, easy OFF feature of our coil design will be extremely useful when scanning sick neonates inside the incubator without significantly disturbing the environment (temperature, humidity, oxygen levels) inside the incubator.

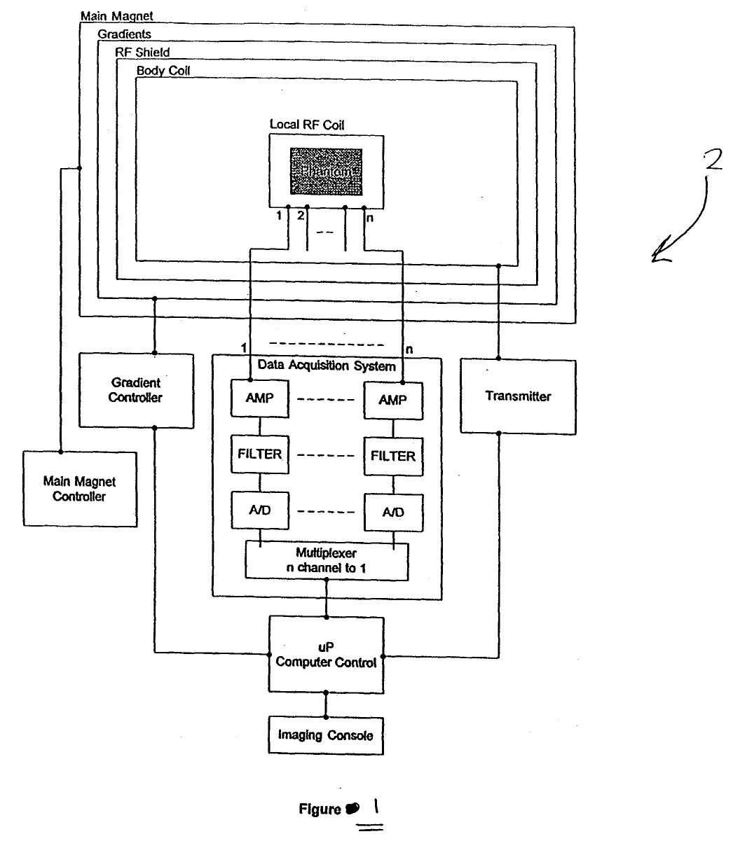

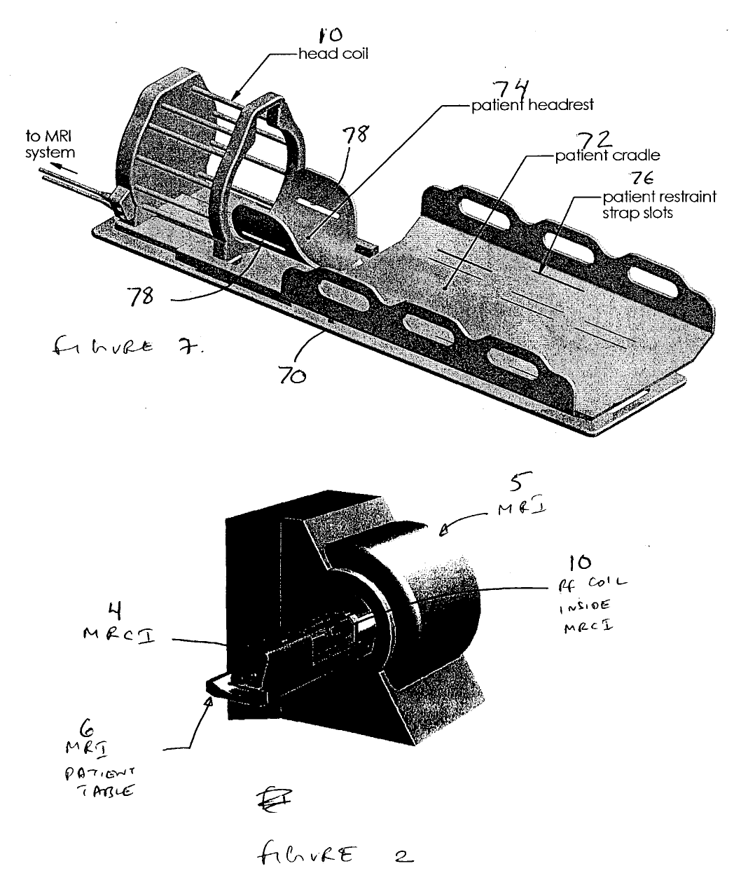

FIG. 1 is a block diagram of an MRI system 2. FIG. 2 is the rendering of the MRCI 4 on the MRI patient table 6. The RF coil 10 can be seen on one end inside the incubator.

Referring to FIG. 3, the preferred embodiment includes an end-capped asymmetric head coil 10 of the low-pass configuration with clearances 12 for the nurse to have access to the patient and while accommodating life sustaining endo-tracheal/ventilator tubes attached to the ill pediatric patient.

The head RF coil 10 has an end-cap 14 similar to the coil of Hayes [6] to improve the field distribution over the brain of the infant. The radial end cap is similar in axial (XY) cross-section to the front of the coil 16 except for an anterior extension 18. The circular end cap extends roughly 1 inch along the coil axis toward to the back which helps minimize the shield currents on the ground of the cables 20 exiting the coil 10 and the incubator system.

The cross-sectional diameter (182 mm) and length (146 mm) of the twelve section RF coil 10 was chosen to include 95th percentile of the patient population up to 3 months of age. The legs 22 in the anterior section are spaced apart azimuthally by 45 degrees to allow a nurse/doctor access to the patient's face. The end ring 24 of the birdcage anterior to the patient is curved in such a way so as to allow placement of life sustaining devices such as the endo-tracheal tube or the ventilator tubes to be attached to the patient at all times (during patient transport between NICU and MRI sections and during MRI scan). Gap width and height of the extension 19, for example, is 51 mm and 37 mm respectively.

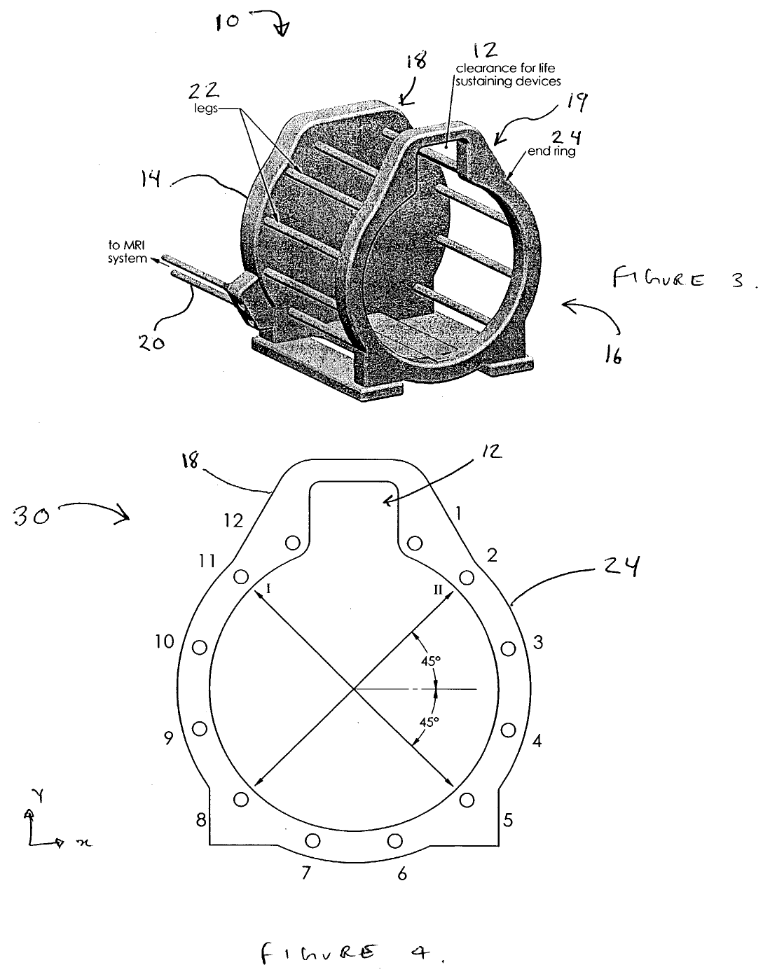

A cross-sectional view 30 of the front end-ring 24 with physical locations of the legs 22 and the alignment of the principal modes (I, II) is shown in FIG. 4. As seen, the principal modes are driven symmetric to the head to realize relatively high but equal Q values and hence optimal S/N.

A planar schematic 40 of the coil 10 of FIG. 3 is shown in FIG. 5. Capacitor values (C1=41.8 pF, C2=40.1 pF, C3=34.1 pF, for example) are chosen such as to provide a sinusoidal current distribution over the coil periphery necessary for providing a high S/N and maintaining a high degree of RF uniformity over the pediatric head. Respective phase shifts are maintained in the coil individual sections based on their physical location, so symmetric mode alignment and drives (a1, a2 and b1, b2) can be realized over the head. This aspect was confirmed by obtaining spin echo images on a homogeneous phantom and on the patient.

High RF shorting capacitors (C4=0.1 uF, for example) are used on the front end ring 24 to eliminate the gradient induced eddy currents. Likewise, the end cap 14 is broken in to smaller sections and several high value RF shorting capacitors are used to bridge the smaller sections (not shown). By doing so, the RF integrity of the end cap 14 is maintained and the coil 10 produces no visible artifacts due to eddy currents usually caused by fast switching time varying gradients.

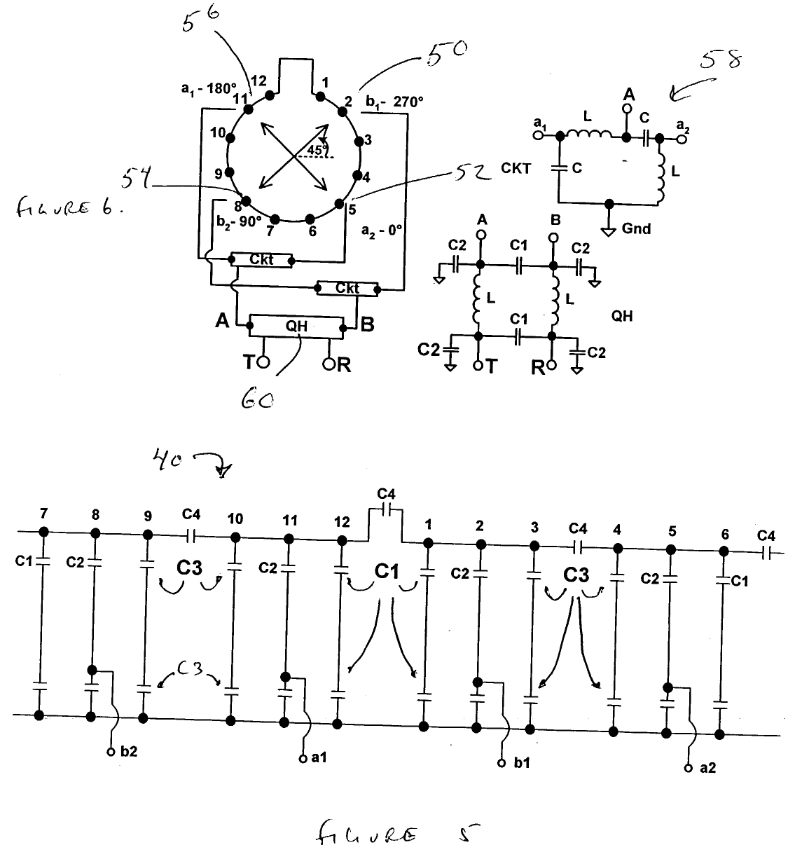

The coil is driven symmetrically at four places ±45 degrees as shown in FIG. 6. This symmetric feed provides a high S/N and a high degree of RF uniformity over the imaging ROI. Four-point feed is achieved using a push-pull configuration as shown in FIG. 6. Drive points a1 and a2, bland b2 are bridged using the push-pull configuration and their outputs are combined with a 3 dB quadrature hybrid combiner 60. Note the characteristic impedance of fifty ohms is used for the phase-shifter push-pull circuit and the quadrature hybrid combiner 60.

During transmit, the power is incident to the coil 10 through the T port of the hybrid combiner 60. This transmit power is split into two (A, B) in the quadrature hybrid and fed to the coil in four places (a1, a2, b1, b2) via the phase-shifter push-pull circuit. During receive, NMR signals from the two linear modes of the coils (1, 11) are combined using the same push-pull circuit and will approach the NMR receive via the R port of the quadrature hybrid 60 owing to the NMR reciprocity principle. Matching the coil to fifty ohms can be accomplished in the four locations a1, a2, b1, b2 by using reactive elements (not shown).

Second Embodiment

Referring to FIG. 7, the second embodiment shows the coil 10 on a base plate 70 that supports the patient in a cradle shaped structure 72 while the patient is being scanned. Note, this set up can be used despite the incubator as a stand alone imaging device.

As shown in FIG. 7, the coil 10 is positioned far back away from the patient head rest 74. The patient is placed on the cradle support 72 (length 664 mm, width 332 mm, height 96 mm, for example) and held with straps via slots 76 if needed to keep the patient still during scanning. The head of the patient can also be held still using a soft velcro strap (not shown) via the two slots 78 shown on the head rest 74 (156 mm long, for example). Note, a soft pad (not shown) can be placed between the patient and the support shown here. Once the patient is placed on the cradle and is cooperative, the RF coil 10 is slid over the head for scanning. Once the scan is finished, the coil 10 is quickly slid back and the patient removed from the MR table. Note, restraining mechanisms such as velcro may be added as needed to hold the head, chest, arms, torso and feet of patients. In addition, some patients may be sedated using doctor prescribed sedatives during transport and the MRI scan. Total length and height of the base plate is 876 and 134 mm, for example.

It is to be noted a simple mechanism is shown as a preferred embodiment. Several transmit/receiver or receive only schemes can be used instead. Further, one or more channels can be used to receive NMR signals in an array configuration. In addition, different configurations of the birdcage coil (high, band pass or stop) can be used. Different sealants and sealing methods also can be used.

Although particular embodiments of the invention have been described in detail, it is understood that the invention is not limited correspondingly in scope, but includes all changes, modifications and equivalents coming within the spirit and terms of the claims appended hereto.

What is claimed is:

1. A head coil, comprising:
an end cap;
an end ring;
a plurality of legs connecting the end cap to the end ring, wherein the plurality of legs are spaced apart azimuthally by 45 degrees; and
a plurality of drive points, wherein the drive points are bridged using a push-pull configuration.

2. The head coil of claim 1, further comprising:
a plurality of outputs, wherein the outputs are combined with a 3 dB quadrature hybrid converter.

3. The head coil of claim 1, where an anterior portion of the end ring includes an access way to allow placement of human diagnostic connections.
